(19) United States
(12) Patent Application Publication
(10) Pub. No.: US 2008/0171927 A1
(43) Pub. Date: Jul. 17, 2008

(54) Physiological detector with a waterproof structure

(75) Inventors: Paul Yang, Taipei (TW); Yi-Tsung Lin, Taipei County (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD., CHUNG HO CITY (TW)

(21) Appl. No.: 11/652,034

(22) Filed: Jan. 11, 2007

(51) Int. Cl. A61B 5/1455

(52) U.S. Cl. 600/340; 600/344

(57) ABSTRACT

A physiological detector with a waterproof structure installed on a glove body corresponding to a finger has a sealed casing for wrapping a detection element. The casing includes a conducting cylinder with an end electrically connected to the detection element and another end exposed from the casing and fixed onto the glove body. The glove body has a conducting circuit electrically connected to an exposed end of the conducting circuit and conducting cylinder, and a physiological measuring device installed at a position corresponding to the back of a hand for processing human physiological parameters detected by the detection element. The physiological measuring device is electrically connected to the physiological detector through the conducting circuit, so that users can wear the physiological detector easily. The detection element of the physiological detector is wrapped into a sealed casing, and thus it is not necessary to remove the detector for a convenient washing.

### BACKGROUND OF THE INVENTION

1. Field of the Invention

The present invention relates to a physiological detector with a waterproof structure, and more particularly to a physiological detector having detection elements sealed into casings and electrically connected to the physiological detector through conducting cylinders and conducting circuits that are integrated into a glove to facilitate users to wear the glove with the physiological detector, and users need not to remove the physiological detector for washing the glove, and thus the invention can save the time for the process of removing the physiological detector.

2. Description of the Related Art

As our living standard improves, people pay more attentions to their health, and thus various different measuring instruments and devices are provided for people to check their physical conditions anytime and make appropriate adjustments and remedial actions, and a common measuring item is the measurement of a heartbeat.

Referring to FIG. 1 for a conventional wearing heartbeat meter, the meter comprises a glove 100, a physiological measuring device 110 installed onto the glove 100 for measuring a heartbeat, and a display screen 111 installed on the physiological measuring device 110 for displaying the heartbeat reading, The physiological measuring device 110 installs a conducting circuit 112 with another end connected to a set of light detector 130 (including a light transmitter and a light receiver), and an end of the glove 100 is extended to an area corresponding to a finger, and the area has a finger cover 120, so that a user can wear the glove 100 onto a palm and sheathe the finger cover 120 onto a finger, and plug the light detector 130 into the finger cover 120. The heartbeat of a human body can be detected by the transmission and reflection of a light source. Since it is necessary to plug the light detector 130 into the finger cover 120, users may find the wearing inconvenient. Furthermore, the physiological measuring device 110 is fixed onto the glove 100 and cannot be washed easily, and thus the application cannot meet user's requirements.

### SUMMARY OF THE INVENTION

In view of the shortcomings of the prior art including the inconvenient wearing and washing of the glove together with the physiological detector, the inventor of the present invention based on years of experience in the related industry to conduct extensive researches and experiments, and finally developed a physiological detector with a waterproof structure in accordance with the present invention to overcome the shortcomings.

Therefore, it is a primary objective of the present invention to provide a physiological detector with a waterproof structure, wherein the physiological detector is installed on a glove body at an area corresponding to a finger, and the physiological detector further includes a sealed casing for wrapping a detection element, and the casing includes a conducting cylinder embedded in the casing and fixed onto the glove body and electrically connected to a conducting circuit and the detection element on the glove body. The glove body further includes a conducting contact disposed at a position corresponding to the back of a hand and connected to a conducting circuit of the glove body, and the conducting contact is electrically connected to physiological measuring device for detecting various physiological parameters of a human body by a detection element and processing the physiological parameters. The physiological measuring device is electrically connected to the conducting circuit and the detection element through the conducting contact, and thus users can wear the glove with the physiological measuring device conveniently for the use of the device. Since the detection element of the physiological detector is wrapped in the sealed casing, it is not necessary to remove the physiological detector for washing the glove and provide a more convenient way for cleaning the globe together with the physiological detector.

Another objective of the present invention is to provide a physiological detector with a waterproof structure, wherein an end of each conducting cylinder is integrated and embedded in the base and another end of the conducting cylinder is exposed from the casing and soldered with the conducting hole of the glove body.

A further objective of the present invention is to provide a physiological detector with a waterproof structure, wherein each conducting cylinder is exploded from an end of the casing and fixed onto a glove body by a conducting fixing element, and both fixing element and conducting cylinder are corresponding rivet and ring.

Another further objective of the present invention is to provide a physiological detector with a waterproof structure, wherein each conducting cylinder is exposed from an end of the base and fixed onto a glove body by a conducting fixing element, and both fixing element and conducting cylinder are corresponding male and female screws.

Another objective of the present invention is to provide a physiological detector with a waterproof structure, wherein the detection element is a light transmitter/receiver for measuring blood oxygen concentration.

Another objective of the present invention is to provide a physiological detector with a waterproof structure, wherein the conducting circuit on the glove body is woven with the fibers of the glove body.

To make it easier for our examiner to understand the objective of the invention, its structure, innovative features, and performance, we use a preferred embodiment together with the attached drawings for the detailed description of the invention

### BRIEF DESCRIPTION OF THE DRAWINGS

FIG. 1 is a schematic view of a conventional device;

FIG. 2 is a schematic perspective view of an application of the present invention;

FIG. 2A is an enlarged view of a physiological detector installed onto a glove body in accordance with the present invention;

FIG. 3 is an exploded view of a physiological detector in accordance with a preferred embodiment of the present invention;

FIG. 4 is a perspective view of a physiological detector connected to a partial area of a glove body in accordance with the present invention; and

FIG. 5 is a cross-sectional view of a physiological detector fixed onto a glove in accordance with the present invention.

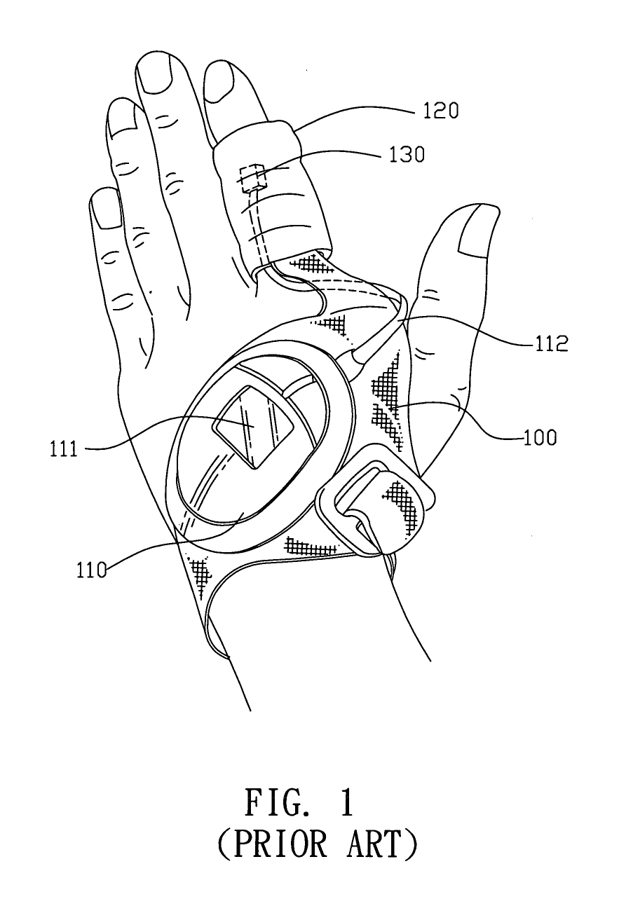

### DETAILED DESCRIPTION OF THE PREFERRED EMBODIMENTS

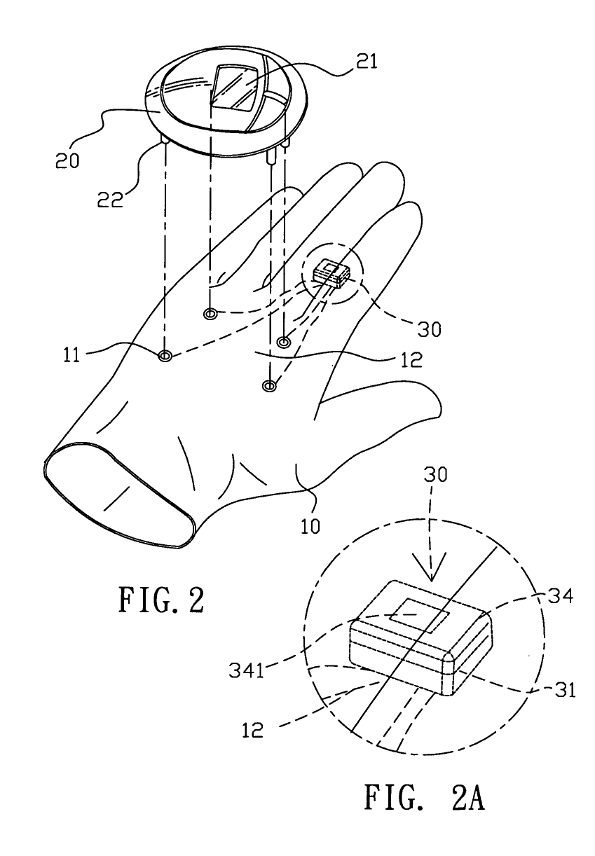

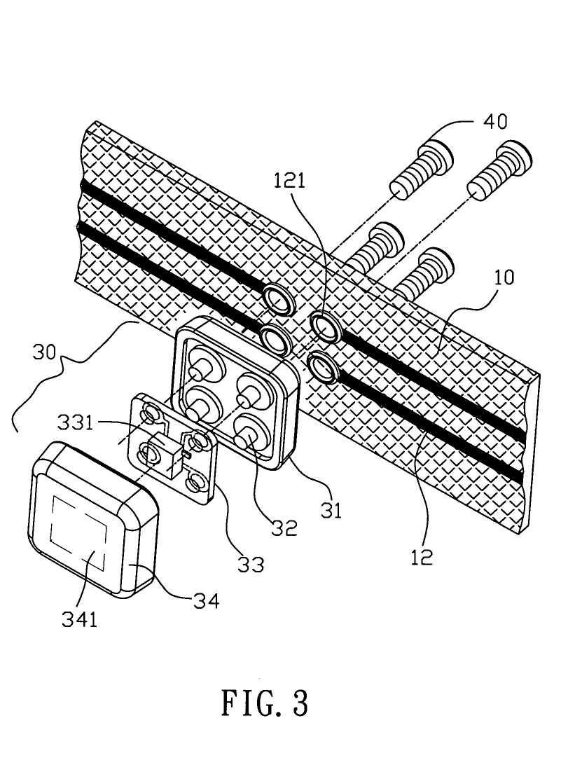

Referring to FIGS. 2, 2A and 3 for a physiological detector with a waterproof structure of the present invention, the physiological detector is installed onto a glove body 10, and the glove body 10 is made of a fiber material for users to wear on their hand, and the glove body 10 has a conducting contact 11 disposed at a position corresponding to the back of a hand, and the conducting contact 11 is made of an electric conducting material. Further, a physiological measuring device 20 is connected to the glove body 10 for processing various detected physiological parameters of a human body. The physiological measuring device 20 further includes a display screen 21 installed on the surface of the physiological measuring device 20 for displaying the detected readings, and the physiological measuring device 20 has an electric connecting element 22 for electrically connecting the conducting contact 11.

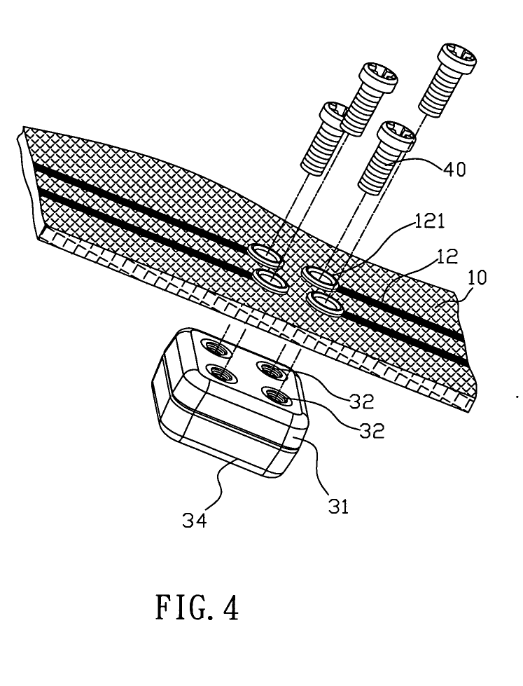

The glove body 10 further includes a conducting circuit 12 disposed at an area corresponding to any finger (which is the middle finger in this embodiment), and the conducting circuit 12 is combined and woven into the glove body 10 by conducting fibers, and the conducting circuit 12 is connected with the conducting contact 11, and the conducting circuit 12 is in contact with the physiological detector 30 (which is a set of a light transmitter and a light receiver in this embodiment for detecting the blood oxygen concentration of human body). The physiological detector 30 has a base 31, and the base 31 has one or more conducting cylinders 32 (which is integrated with the base by injection molding in this embodiment), and an end of the conducting cylinder 32 is exposed from the base 31 (which is an inwardly extended female screw in this embodiment), and another end of the conducting cylinder 32 is electrically contacted with the detection element 33 on the base 31, and the detection element 33 installs a light transmitter/receiver 331, and a contact 332 electrically and precisely connected with the conducting cylinder 32, and a cover 34 is covered onto the base 31. The cover 34 is sealed with the base 31 by ultrasonic welding to form a sealed casing for preventing outside moisture from entering into the sealed casing and protecting the detection element 33 inside the casing. The cover 34 has a transparent region 341 (as shown in FIGS. 3 and 4) corresponding to the light transmitter/receiver 331 to facilitate the light transmission and receiving of a light transmitter/receiver.

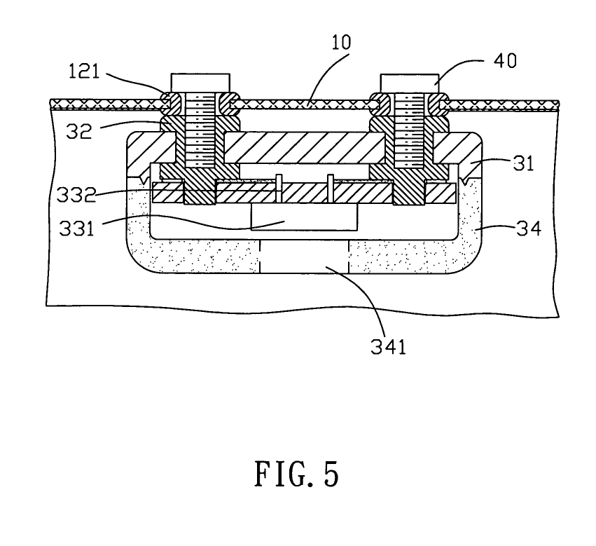

In addition, the conducting circuit 12 on the glove body 10 has at least one metal conducting hole 121 disposed at a position corresponding to the physiological detector 30, and the metal conducting hole 121 is sheathed with a fixing element 40 (which is a male screw corresponding to the conducting cylinder in this embodiment, but those skilled in the art can adopt corresponding rivet and ring to connect the conducting cylinder or directly solder the conducting cylinder with the conducting hole), and the fixing element 40 can be connected to the conducting cylinder 32 through the metal conducting hole 121, such that the physiological detector 30 is fixed onto the surface of the glove body 10 for electrically connecting the conducting circuit 12 (as shown in FIG. 5).

Referring to FIGS. 2, 4 and 5, users need not to remove the physiological detector 30 from the glove body 10 for washing the glove body 10, since the physiological detector 30 is wrapped and sealed by the sealed casing, and such arrangement makes the application very simple and easily and saves much time.

In summation of the above description, the physiological detector with a waterproof structure of the present invention comes with a simple structure and allows users to obtain accurate measurements of their physical conditions anytime, so that users can make appropriate adjustment and actions for their health. Therefore, the invention herein enhances the performance than the conventional structure and further complies with the patent application requirements, and thus is duly filed for patent application.

What is claimed is:

1. A physiological detector with a waterproof structure detection component, installed on a glove body and said glove body having a conducting hole and a conducting circuit electrically connected to said conducting hole, and said physiological detector comprising:
a base;
a cover, sealed onto said base to form a sealed casing;
a detection element, installed in said casing comprised of said base and said cover;
one or more conducting cylinders, embedded into said base, and an end of said each conducting cylinder being electrically connected to said detection element, and another end of said each conducting cylinder being exposed from said base and fixed onto said glove body and electrically connected with said conducting hole;
thereby, said sealed casing is formed by engaging said cover and said base, for preventing outside moisture from entering into said sealed casing and protecting said detection element.

2. The physiological detector with a waterproof structure of claim 1, wherein said glove body includes a physiological measuring device installed thereon and electrically connected to said physiological detector through a conducting circuit.

3. The physiological detector with a waterproof structure of claim 2, wherein said conducting circuit is mixed and woven into said glove body in advance.

4. The physiological detector with a waterproof structure of claim 1, wherein said conducting cylinder is exposed from an end of said base and soldered with a conducting hole disposed on said glove body.

5. The physiological detector with a waterproof structure of claim 1, wherein said conducting cylinder has a conducting fixing element exposed at an end of said base, and said conducting cylinder has a conducting fixing element disposed at an exposed end of said conducting cylinder, and both fixing elements are corresponding rivet and ring, and passed through said conducting hole, and fixed onto said glove body.

6. The physiological detector with a waterproof structure of claim 1, wherein said conducting cylinder includes a conducting male screw and a conducting female screw corresponding to each other, and both conducting male and female screws are disposed at an exposed end of said conducting cylinder, passed through said conducting hole, and fixed onto said glove body.

7. The physiological detector with a waterproof structure of claim 1, wherein said physiological detector is a light transmitter/receiver for measuring blood oxygen concentration.

8. The physiological detector with a waterproof structure of claim 7, wherein said cover includes a transparent region for facilitating light transmission and receiving of said light transmitter/receiver.
